(19) United States
(12) Patent Application Publication
(10) Pub. No.: US 2022/0197252 A1
(43) Pub. Date: Jun. 23, 2022

(54) METHODS, MEDIUMS, AND SYSTEMS FOR STORING AND RETRIEVING CHROMATOGRAPHY DATA

(71) Applicant: WATERS TECHNOLOGIES IRELAND LIMITED, Dublin 2 (IE)

(72) Inventors: Chris Jensen, Coventry, CT (US); Steven M. Cohn, Franklin, MA (US)

(21) Appl. No.: 17/559,013

(22) Filed: Dec. 22, 2021

Related U.S. Application Data
(60) Provisional application No. 63/130,058, filed on Dec. 23, 2020.

(51) Int. Cl. G05B 19/4155

(52) U.S. Cl. CPC G05B 19/4155; G05B 2219/37583

(57) ABSTRACT

Exemplary embodiments provide methods, mediums, and systems for providing services and systems for managing data in a chromatography data processing environment. The system may implement one or more autonomous services that read encoded data and decode the data for use by one or more applications. To communicate with the autonomous services, the autonomous services may expose endpoints accessible to requesting applications. For instance, the endpoints may apply decoders to read a stream of data generated by a chromatography instrument. Each of the decoders may be configured to read different configurations of data (e.g., to parse the stream based on the way that the instrument encodes the stream). Accordingly, the acquired data from many different instruments can be stored in a centralized repository, and may be accessed by any authorized application through the endpoints. The data processing environment therefore serves as a single access point for many different types of data.

### CROSS-REFERENCE TO RELATED APPLICATIONS

This application claims the benefit of U.S. Provisional Patent Application No. 63/130,058, filed Dec. 23, 2020. The entire disclosure of which is hereby incorporated by reference.

### BACKGROUND

Chromatography refers to the separation of a mixture by passing it in solution, suspension, or as a vapor through a medium in which the components of the mixture move at different rates. The components may then be analyzed to identify the existence, amount, concentration, or other properties of the components. Chromatography includes a number of different techniques, such as mass spectrometry (MS), liquid chromatography mass spectrometry (LCMS), and many others.

### BRIEF SUMMARY

Exemplary embodiments relate to techniques for retrieving and processing chromatography data in a data processing environment. Unless otherwise noted, it is contemplated that these embodiments may be used individually in order to achieve the advantages noted, or in any combination in order to achieve synergistic effects.

According to a first embodiment, a chromatography data processing environment including one or more autonomous services may be provided. An autonomous service may be a computing service that is independent of other services for its functionality. Although it may asynchronously provide information that may be consumed by other services (and may asynchronously consume information from other services), autonomous services may refrain from assisting other services synchronously through behaviors.

Each of the autonomous services may expose one or more endpoint interfaces. For example, these endpoints may Representation State Transfer (REST) endpoints capable of receiving RESTful Application Programming Interface (API) calls. An endpoint interface may receive a request for raw data acquired by a chromatography instrument. The data processing environment may expose multiple endpoint interfaces; for example, each autonomous service may be associated with and may expose at least one endpoint interface.

A decoder may be associated with the endpoint interface. The decoder may be configured to interpret the raw data that is associated with the endpoint interface. For example, the endpoint interface may be associated with a particular type of raw data generated by a chromatography instrument in a specific acquisition mode. That instrument may output a stream of raw data, including (e.g.) binary data, arrays of information, etc. The decoder may be programmed to parse a stream of raw data generated by such an instrument so that the data stream can be meaningfully interpreted.

The autonomous service (or another construct) may retrieve the requested raw data from a raw data store, apply the decoder to the raw data to generate decoded data, and may return the decoded data in response to the original request. For example, the autonomous service may apply the decoder to the raw data and provide decoded data to the requesting application, or the autonomous service may identify the decoder and provide it (or a location at which it can be accessed) to the requesting application along with the raw data (or a location of the raw data). In the latter case, the application may decode the data with the decoder.

By exposing the endpoint interfaces in this way, an application can request data acquired by a chromatography instrument without needing to understand how to interpret the data. Accordingly, when new types of instruments are brought online (potentially outputting data in a different streaming format), it is not necessary to reprogram each application that might use that data. Because each application need not be programmed with specifics of how to interpret each different type of data stream, more different types of data can be made available to the applications, which allows for more complex analyses. This configuration also allows multiple different types of data to be stored together in a common source structure, simplifying data retrieval and storage.

According to a second embodiment, the autonomous service may be a first autonomous service configured to interface with the raw data store. A second autonomous service may be provided, where the second autonomous service is configured to interface with a metadata catalog storing metadata that describes how the raw data was acquired.

In this configuration, multiple autonomous services service incoming requests for data. At least one of the autonomous services is specifically configured to respond to requests for metadata, while the remaining autonomous services respond to requests for different types of data. The autonomous service responding to metadata requests does not respond to requests for data, and accordingly does not need to implement any functionality related to the decoders. Similarly, the autonomous services responding to data requests do not need to implement any of the functionality for querying the metadata catalog. When new data types are added, a new autonomous service implementing the decoder for the new data type may be added, or an existing autonomous service may be updated with the new functionality. Meanwhile, most of the autonomous services can remain unchanged. Similarly, if the metadata catalog API is ever changed, only the metadata-handling autonomous service needs to be updated.

According to a third embodiment, the endpoint interface may be a first endpoint interface and is configured to receive requests for raw data. A second endpoint interface separate from the first endpoint interface may be provided, where the second endpoint interface may be configured to receive requests for metadata that describes how the raw data was acquired.

In this embodiment, incoming requests are separated into metadata-specific requests and data-specific requests. Each is handled by a different type of endpoint. The third embodiment may be combined with the second embodiment, so that the metadata-handling autonomous service implements the second endpoint interface, and the data-handling autonomous service(s) implement copies of the first endpoint interface, which helps to segregate incoming requests and provides requesting applications with a known endpoint to target for appropriate types of requests. This embodiment may also be deployed separately, where a single autonomous service implements both the metadata-specific second endpoint interface and the data-specific first endpoint interface. Multiple copies of such an autonomous service may be provided, where each implements both the metadata endpoint interface and the data endpoint interface(s). It is relatively straightforward to implement both a metadata-specific endpoint and data-specific endpoint(s) on a single autonomous service, and this embodiment allows for a straightforward scale-out process (i.e., additional capacity can be implemented by providing additional copies of autonomous service).

According to a fourth embodiment, a single decoder may be associated with multiple different endpoint interfaces. This allows decoders to be recycled to interpret data streams that may have originated with different chromatography apparatus configurations, but which provide raw data formatted in the same way. Similarly, a single decoder might have multiple versions, each of which map to a different endpoint (similarly, different decoders for the same acquisition mode may be used if the acquisition mode can capture different types of data).

According to a fifth embodiment the raw data store may include data from multiple different chromatography apparatuses and/or chromatography apparatuses operating in multiple different acquisition modes. In the fifth embodiment, the data processing environment acts as a single source of data for applications, regardless of which device generated the data (or which mode the data was operating in). Any application calling into the ecosystem can be sure that any acquired data can be accessed and processed appropriately.

According to a sixth embodiment, the raw data store includes data of a multiple different data types, and the autonomous service is one of a plurality of autonomous services. Collectively, the autonomous services may be configured to decode each of the plurality of different data types. For example, the multiple different data types may be included in an interface specification, which may describe how to decode the various different types. The interface specification may be capable of being implemented, at least in part, by each of the autonomous services by implementing corresponding endpoint interfaces and decoders. Different parts of the interface specification may be split between multiple different autonomous services, so that each implements a part, but not all, of the interface specification. Each part of the interface specification may be implemented by at least one of the autonomous services so that, collectively, the group of interface services implements the interface specification.

Because each autonomous service is tasked with only implementing a portion of the interface specification, each autonomous service can be made simpler (since it need not be concerned with providing decoders and endpoint interfaces for portions of the interface specification that it does not implement). New autonomous services can be easily added to deal with new capabilities, and it is not necessary to take down all of the autonomous services when one decoder needs to be updated.

According to a seventh embodiment based on the sixth embodiment, the plurality of autonomous services may collectively include a plurality of endpoint interfaces, each endpoint interface characterized by a data type. When an application requests data, it may call into the raw data store or the metadata catalog to identify the type of the data; for example, the data may be tagged with a codec key that is stored with the data and/or in the metadata. The endpoint interfaces may be callable based on the data type, so once the data type is known the requesting application may identify the appropriate endpoint interface to decode the data and may formulate an appropriate RESTful API call to communicate with the interface. This provides an efficient way for the application to identify and call into the autonomous service that is capable of decoding the data.

These embodiments will be described in detail below with reference to the accompanying Figures.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an example of a mass spectrometry system according to an exemplary embodiment.

FIG. 2 illustrates an example of a workflow that operates on acquired data in accordance with one embodiment.

FIG. 3A illustrates a first exemplary data processing ecosystem in accordance with one embodiment.

FIG. 3B illustrates a second exemplary data processing ecosystem in accordance with another embodiment.

FIG. 4 is a flowchart depicting exemplary logic for storing and retrieving chromatography data in accordance with an exemplary embodiment.

FIG. 5 depicts exemplary logic for adding a new decoder to a data processing environment in accordance with one embodiment.

FIG. 6 depicts an illustrative computer system architecture that may be used to practice exemplary embodiments described herein.

### DETAILED DESCRIPTION

The data acquired by chromatography instruments can be highly complex. Different types of instruments, operating in different modes and performing different experiments, may generate data in many different formats. That data may then need to be analyzed for several different purposes, generating multiple different types of output data. In conventional solutions, each raw data stream would be stored in a raw data file, and each application processing the data would store processed data in its own application-specific format and location. In some cases, input raw data may be distributed to many different locations, and the same input raw data might be unnecessarily duplicated. Moreover, a user may need to know where the data is stored and may need to program each analysis program to be able to interpret the data. Because of the need to program each application to learn to recognize different types of input data, in many cases applications are only configured to interpret a minimal subset of data types. This may limit the types of analyses that can be performed.

Exemplary embodiments described herein address these and other issues by providing services and systems for managing data in a chromatography data processing environment.

The system may implement one or more autonomous services that read encoded data and decode the data for use by one or more applications. To communicate with the autonomous services, the autonomous services may expose endpoints accessible to requesting applications. For instance, the endpoints may be RESTful endpoints that apply decoders to read a stream of data generated by a chromatography instrument. Each of the decoders may be configured to read different configurations of data (e.g., to parse the stream based on the way that the instrument encodes the stream). Accordingly, the acquired data from many different instruments can be stored in a centralized repository, and may be accessed by any authorized application through the endpoints. Accordingly, the data processing environment serves as a single access point for many different types of data.

The environment may include multiple autonomous services, each configured for particular purposes. For instance, one or more of the autonomous services may be configured to retrieve metadata associated with a particular stream of raw data; the metadata may indicate a type of the raw data, a decoder that can decode the raw data, and other information about the acquisition of the data (e.g., vial amounts, sample components lists, etc.). An application can call into the endpoint associated with the metadata autonomous service to retrieve this information. Another set of autonomous services may be configured with decoders designed to parse the raw data (which is generally in the form of a stream) and return a set of parsed data (e.g., a chromatography sample set). In some embodiments, multiple autonomous services may be provided to decode data, where each autonomous service is responsible for a relatively small number of data types (a subset that is smaller than the full set of data types). Thus, each autonomous service only needs to implement a relatively small number of endpoints and can be made more efficient to load into memory and run.

These and other features will be described in more detail below with reference to the accompanying figures.

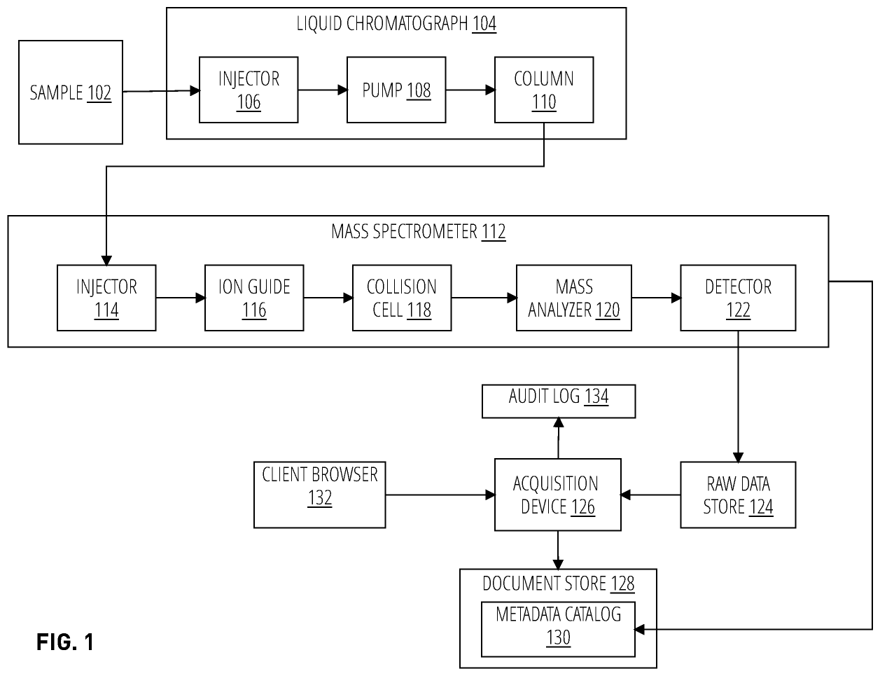

For purposes of illustration, FIG. 1 is a schematic diagram of a system that may be used in connection with techniques herein. Although FIG. 1 depicts particular types of devices in a specific LCMS configuration, one of ordinary skill in the art will understand that different types of chromatographic devices (e.g., MS, tandem MS, etc.) may also be used in connection with the present disclosure.

A sample 102 is injected into a liquid chromatograph 104 through an injector 106. A pump 108 pumps the sample through a column 110 to separate the mixture into component parts according to retention time through the column.

The output from the column is input to a mass spectrometer 112 for analysis. Initially, the sample is desolved and ionized by a desolvation/ionization device 114. Desolvation can be any technique for desolvation, including, for example, a heater, a gas, a heater in combination with a gas or other desolvation technique. Ionization can be by any ionization techniques, including for example, electrospray ionization (ESI), atmospheric pressure chemical ionization (APCI), matrix assisted laser desorption (MALDI) or other ionization technique. Ions resulting from the ionization are fed to a collision cell 118 by a voltage gradient being applied to an ion guide 116. Collision cell 118 can be used to pass the ions (low-energy) or to fragment the ions (high-energy).

Different techniques (including one described in U.S. Pat. No. 6,717,130, to Bateman et al., which is incorporated by reference herein) may be used in which an alternating voltage can be applied across the collision cell 118 to cause fragmentation. Spectra are collected for the precursors at low-energy (no collisions) and fragments at high-energy (results of collisions).

The output of collision cell 118 is input to a mass analyzer 120. Mass analyzer 120 can be any mass analyzer, including quadrupole, time-of-flight (TOF), ion trap, magnetic sector mass analyzers as well as combinations thereof. A detector 122 detects ions emanating from mass analyzer 122. Detector 122 can be integral with mass analyzer 120. For example, in the case of a TOF mass analyzer, detector 122 can be a microchannel plate detector that counts intensity of ions, i.e., counts numbers of ions impinging it.

A raw data store 124 may provide permanent storage for storing the ion counts for analysis. For example, raw data store 124 can be an internal or external computer data storage device such as a disk, flash-based storage, and the like. An acquisition device 126 analyzes the stored data. Data can also be analyzed in real time without requiring storage in a storage medium 124. In real time analysis, detector 122 passes data to be analyzed directly to computer 126 without first storing it to permanent storage.

Collision cell 118 performs fragmentation of the precursor ions. Fragmentation can be used to determine the primary sequence of a peptide and subsequently lead to the identity of the originating protein. Collision cell 118 includes a gas such as helium, argon, nitrogen, air, or methane. When a charged precursor interacts with gas atoms, the resulting collisions can fragment the precursor by breaking it up into resulting fragment ions. Such fragmentation can be accomplished as using techniques described in Bateman by switching the voltage in a collision cell between a low voltage state (e.g., low energy, <5 V) which obtains MS spectra of the peptide precursor, with a high voltage state (e.g., high or elevated energy, >15V) which obtains MS spectra of the collisionally induced fragments of the precursors. High and low voltage may be referred to as high and low energy, since a high or low voltage respectively is used to impart kinetic energy to an ion.

Various protocols can be used to determine when and how to switch the voltage for such an MS/MS acquisition. For example, conventional methods trigger the voltage in either a targeted or data dependent mode (data-dependent analysis, DDA). These methods also include a coupled, gas-phase isolation (or pre-selection) of the targeted precursor. The low-energy spectra are obtained and examined by the software in real-time. When a desired mass reaches a specified intensity value in the low-energy spectrum, the voltage in the collision cell is switched to the high-energy state. The high-energy spectra are then obtained for the pre-selected precursor ion. These spectra contain fragments of the precursor peptide seen at low energy. After sufficient high-energy spectra are collected, the data acquisition reverts to low-energy in a continued search for precursor masses of suitable intensities for high-energy collisional analysis.

Different suitable methods may be used with a system as described herein to obtain ion information such as for precursor and product ions in connection with mass spectrometry for an analyzed sample. Although conventional switching techniques can be employed, embodiments may also use techniques described in Bateman which may be characterized as a fragmentation protocol in which the voltage is switched in a simple alternating cycle. This switching is done at a high enough frequency so that multiple high- and multiple low-energy spectra are contained within a single chromatographic peak. Unlike conventional switching protocols, the cycle is independent of the content of the data. Such switching techniques described in Bateman, provide for effectively simultaneous mass analysis of both precursor and product ions. In Bateman, using a high- and low-energy switching protocol may be applied as part of an LC/MS analysis of a single injection of a peptide mixture. In data acquired from the single injection or experimental run, the low-energy spectra contains ions primarily from unfragmented precursors, while the high-energy spectra contain ions primarily from fragmented precursors. For example, a portion of a precursor ion may be fragmented to form product ions, and the precursor and product ions are substantially simultaneously analyzed, either at the same time or, for example, in rapid succession through application of rapidly switching or alternating voltage to a collision cell of an MS module between a low voltage (e.g., generate primarily precursors) and a high or elevated voltage (e.g. generate primarily fragments) to regulate fragmentation. Operation of the MS in accordance with the foregoing techniques of Bateman by rapid succession of alternating between high (or elevated) and low energy may also be referred to herein as the Bateman technique and the high-low protocol.

The data acquired by the high-low protocol allows for the accurate determination of the retention times, mass-to-charge ratios, and intensities of all ions collected in both low- and high-energy modes. In general, different ions are seen in the two different modes, and the spectra acquired in each mode may then be further analyzed separately or in combination. The ions from a common precursor as seen in one or both modes will share the same retention times (and thus have substantially the same scan times) and peak shapes. The high-low protocol allows the meaningful comparison of different characteristics of the ions within a single mode and between modes. This comparison can then be used to group ions seen in both low-energy and high-energy spectra.

In summary, such as when operating the system using the Bateman technique, a sample 102 is injected into the LC/MS system. The LC/MS system produces two sets of spectra, a set of low-energy spectra and a set of high-energy spectra. The set of low-energy spectra contain primarily ions associated with precursors. The set of high-energy spectra contain primarily ions associated with fragments. These spectra are stored in a raw data store 124. After data acquisition, these spectra can be extracted from the raw data store 124 and displayed and processed by post-acquisition algorithms in the acquisition device 126.

Metadata describing various parameters related to data acquisition may be generated alongside the raw data. This information may include a configuration of the liquid chromatograph 104 or mass spectrometer 112 (or other chromatography apparatus that acquires the data), which may define a data type. An identifier (e.g., a key) for a codec that is configured to decode the data may also be stored as part of the metadata and/or with the raw data. The metadata may be stored in a metadata catalog 130 in a document store 128.

The acquisition device 126 may operate according to a workflow, providing visualizations of data to an analyst at each of the workflow steps and allowing the analyst to generate output data by performing processing specific to the workflow step. The workflow may be generated and retrieved via a client browser 132. As the acquisition device 126 performs the steps of the workflow, it may read read raw data from a stream of data located in the raw data store 124. As the acquisition device 126 performs the steps of the workflow, it may generate processed data that is stored in a metadata catalog 130 in a document store 128; alternatively or in addition, the processed data may be stored in a different location specified by a user of the acquisition device 126. It may also generate audit records that may be stored in an audit log 134.

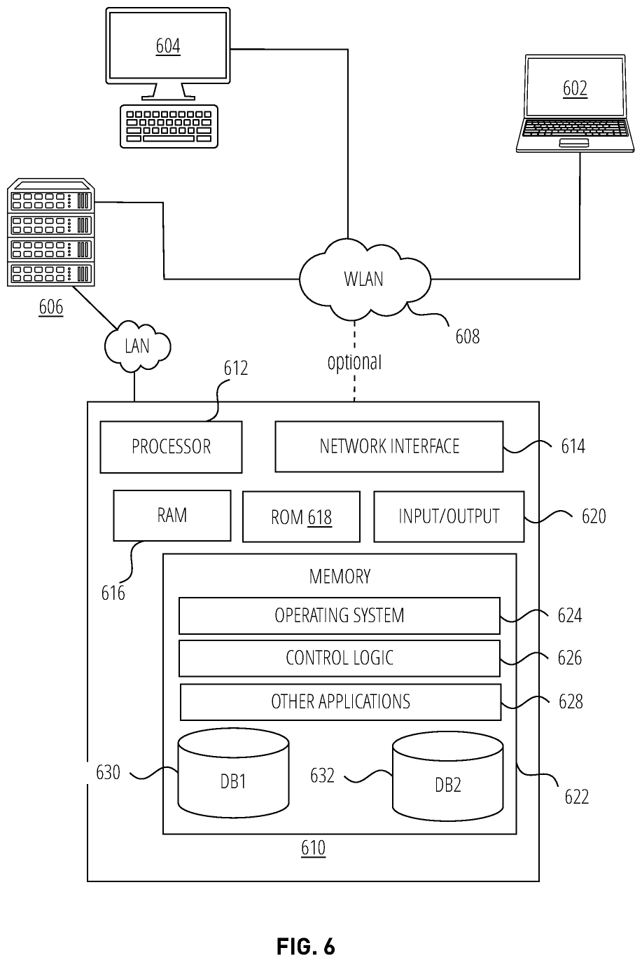

The exemplary embodiments described herein may be performed at the client browser 132 and acquisition device 126, among other locations. An example of a device suitable for use as an acquisition device 126 and/or client browser 132, as well as various data storage devices, is depicted in FIG. 6.

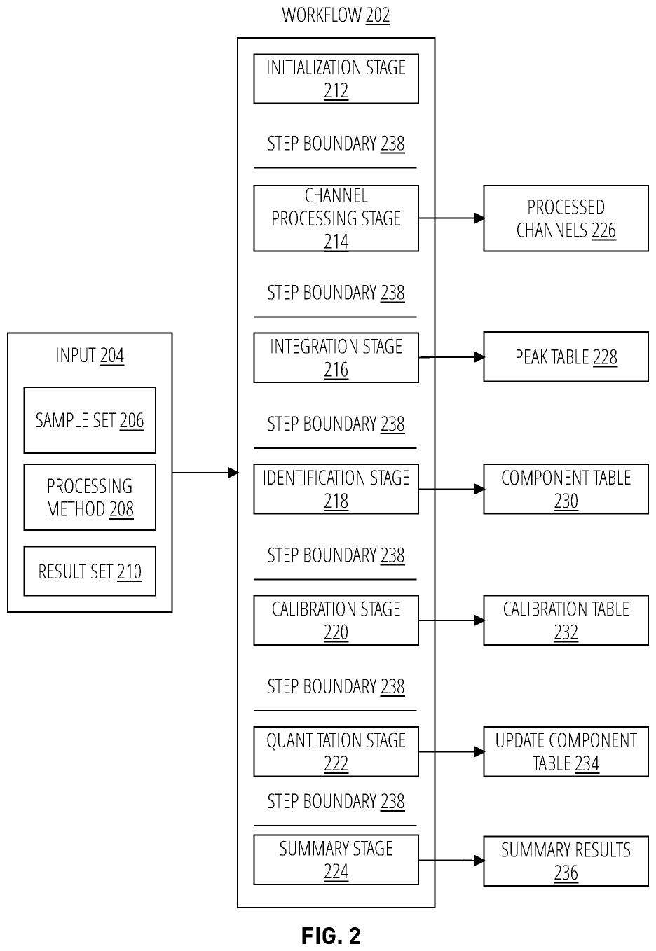

For context, FIG. 2 depicts a simplified example of a workflow 202 that may be applied by the acquisition device 126 of FIG. 1. The workflow 202 is designed to take a set of inputs 204, apply a number of workflow steps or stages to the inputs to generate outputs at each stage, and continue to process the outputs at subsequent stages in order to generate results of the experiment. It is noted that the workflow 202 is a specific example of a workflow, and includes particular stages performed in a particular order. However, the present invention is not limited to the specific workflow depicted in FIG. 2. Other suitable workflows may have more, fewer, or different stages performed in different orders.

The initial set of inputs 204 may include a sample set 206, which includes the raw (unprocessed) data received from the chromatography experimental apparatus. This may include measurements or readings, such as mass-to-charge ratios. The measurements that are initially present in the sample set 206 may be measurements that have not been processed, for example to perform peak detection or other analysis techniques. The sample set 206 may include data in the form of a stream (e.g., a sequential list of data values received in a steady, continuous flow from an experimental apparatus).

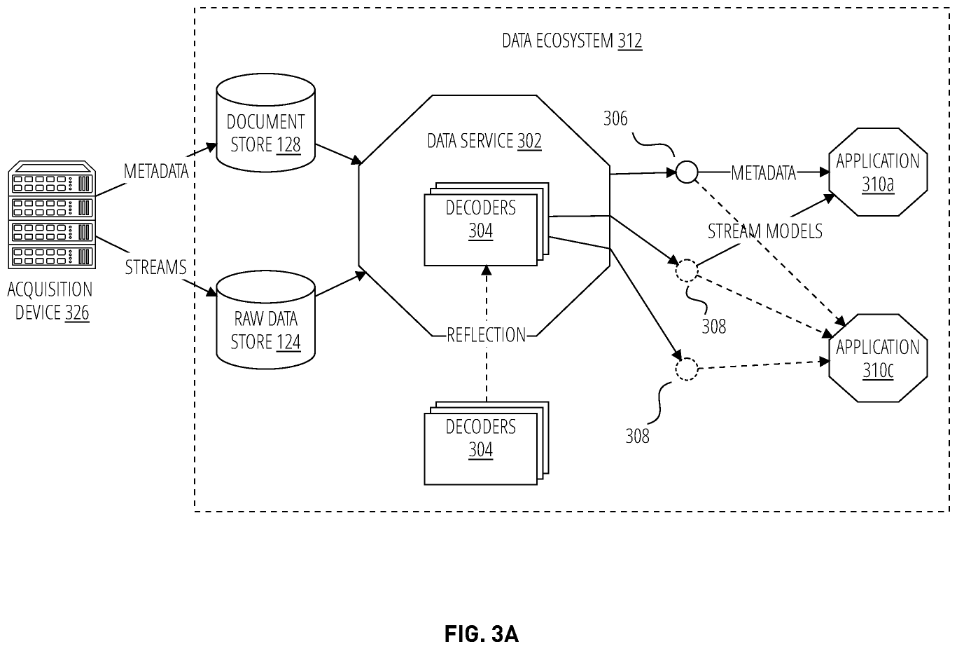

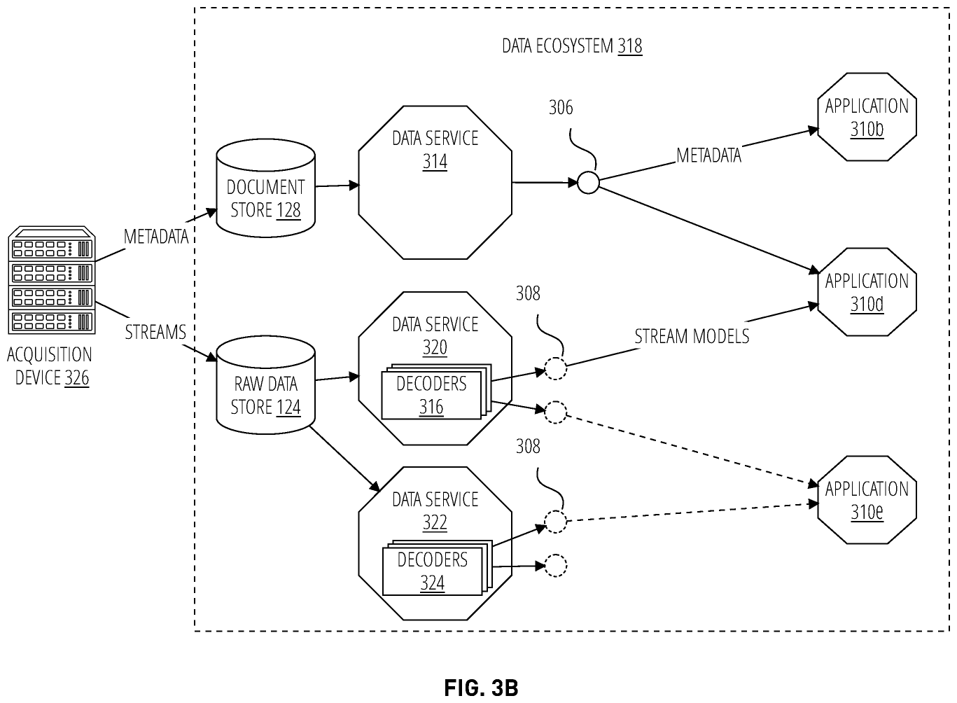

In the context of the present application, the sample set 206 may represent the raw data stored in the raw data store 124 and returned by the endpoint interface. The sample set 206 may be represented as a model of a data stream (e.g., including data structures corresponding to data points gathered by the chromatography apparatus). The workflow 202 may be performed on the sample set 206 data by an application running on the acquisition device 126 and/or running within a data ecosystem 312, as shown in FIG. 3A and FIG. 3B.

The initial set of inputs 204 may also include a processing method 208, which may be a template method (as discussed above) that is applied to (and hence embedded in) the workflow 202. The processing method 208 may include settings to be applied at various stages of he workflow 202.

The initial set of inputs 204 may also include a result set 210. When created, the result set 210 may include the information from the sample set 206. In some cases, the sample set 206 may be processed in some initial manner when copied into the result set 210—for example, MS data may require extracting, smoothing, etc. before being provided to a workflow 202. The processing applied to the initial result set 210 may be determined on a case-by-case basis based on the workflow 202 being used. Once the raw data is copied from a sample set 206 to create a result set 210, that result set 210 may be entirely independent from the sample set 206 for the remainder of its lifecycle.

The workflow 202 may be divided into a set of stages. Each stage may be associated with one or more stage processors that perform calculations related to that stage. Each stage processor may be associated with stage settings that affect how the processor generates output from a given input.

Stages may be separated from each other by step boundaries 238. The step boundaries 238 may represent points at which outputs have been generated by a stage and stored in the result set, at which point processing may proceed to the next stage. Some stage boundaries may require certain types of input in order to be crossed (for example, the data generated at a given stage might need to be reviewed by one or more reviewers, who need to provide their authorization in order to cross the step boundary 238 to the next stage). Step boundaries 238 may apply any time a user moves from one stage to a different stage, in any direction. For example, a step boundary 238 exists when a user moves from the initialization stage 212 to the channel processing stage 214, but also exists when a user attempts to move backwards from the quantitation stage 222 back to the integration stage 216. Step boundaries 238 may be ungated, meaning that once a user determines to move to the next stage no further input (or only a cursory input) is required, or gated, meaning that the user must provide some sort of confirmation indicating that they wish to proceed to a selected stage (perhaps in response to a warning raised by the acquisition device 126), or a reason for moving to a stage, or credentials authorizing the workflow 202 to proceed to the selected stage.

In an initialization stage 212, each of the stage processors may respond by clearing the results that it generates. For example, the stage processor for the channel processing stage 214 may clear all its derived channels and peak tables (see below). At any point in time, clearing a stage setting may clear stage tracking from the current stage and any subsequent stage. In this example, the initialization stage 212 does not generate any output.

After crossing a step boundary 238, processing may proceed to a channel processing stage 214. As noted above, chromatography detectors may be associated with one or more channels on which data may be collected. At the channel processing stage 214, the acquisition device 126 may derive a set of processing channels present in the data in the result set 210, and may output a list of processed channels 226. The list of processed channels 226 may be stored in a versioned sub-document associated with the channel processing stage 214, which may be included in the result set 210.

After crossing a step boundary 238, processing may proceed to an integration stage 216, which identifies peaks in the data in the result set 210 based on the list of processed channels 226. The integration stage 216 may identify the peaks using techniques specified in the settings for the integration stage 216, which may be defined in the processing method 208. The integration stage 216 may output a peak table 228 and store the peak table 228 in a versioned sub-document associated with the integration stage 216. The sub-document may be included in the result set 210.

After crossing a step boundary 238, processing may proceed to identification stage 218. In this stage, the acquisition device 126 may identify components in the mixture analyzed by the chromatography apparatus based on the information in the peak table 228. The identification stage 218 may output a component table 230, which includes a list of components present in the mixture. The component table 230 may be stored in a versioned sub-document associated with the identification stage 218. The sub-document may be included in the result set 210.

After crossing a step boundary 238, processing may proceed to calibration stage 220. During a chromatography experiment, calibration compounds may be injected into the chromatography apparatus. This process allows an analyst to account for subtle changes in electronics, cleanliness of surfaces, ambient conditions in the lab, etc. throughout an experiment. In the calibration stage 220, data obtained with respect to these calibration compounds is analyzed and used to generate a calibration table 232, which allows the acquisition device 126 to make corrections to the data to ensure that it is reliable and reproducible. The calibration table 232 may be stored in a versioned sub-document associated with the calibration stage 220. The sub-document may be included in the result set 210.

After crossing a step boundary 238, processing may proceed to quantitation stage 222. Quantitation refers to the process of determining a numerical value for the quantity of an analyte in a sample. The acquisition device 126 may use the results from the previous stages in order to quantify the components included in the component table 230. The quantitation stage 222 may update 234 the component table 230 stored in the result set 210 with the results of quantitation. The updated component table 230 may be stored in a versioned sub-document associated with the quantitation stage 222. The sub-document may be included in the result set 210.

After crossing a step boundary 238, processing may proceed to summary stage 224. In the summary stage 224, the results of each of the previous stages may be analyzed and incorporated into a report of summary results 236. The summary results 236 may be stored in a versioned sub-document associated with the summary stage 224. The sub-document maybe included in the result set 210.

As used herein, a step may correspond to the above-noted stages. Alternatively, a single stage may include multiple steps, or multiple stages may be organized into a single step. In any event, all the activities performed in a given step should be performable by the same user or group of users, and each step is associated with one or more pages that describe a set of configuration options for the step (e.g., visualization options, review options, step configuration settings, etc.)

There may be a transition at some or all of the step boundaries 238, although not every step boundary 238 need be a transition. A transition may signify a change in responsibility for a set of data from a first user or group of users to a second, distinct user or group of users.

FIG. 3A depicts an exemplary data ecosystem 312 for storing and retrieving chromatography data.

A chromatography acquisition device 326, such as a spectrometer, chromatography, or other device, may perform and output measurements (e.g., as a stream of readings formatted according to a data type that is specific to the acquisition device 326 and/or settings applied to the acquisition device 326). Those measurements may be stored in a raw data store 124.

In one example, the acquisition device 326 may acquire samples using an acquisition controller service. The acquisition controller service may submit the samples, via a RESTful API call, to an acquired data receiver autonomous service. The acquired data receiver autonomous service may create a sample set, which represents the multiple samples sent for analysis into an instrument. In other words, a sample set is an organized sequence of several injections that were sent into the chromatography apparatus.

The raw data raw data store 124 may include data from multiple different chromatography apparatuses and/or chromatography apparatuses operating in multiple different acquisition modes. Accordingly, the data processing environment acts as a single source of data for applications, regardless of which device generated the data (or which mode the data was operating in). Any application calling into the ecosystem can be sure that any acquired data can be accessed and processed appropriately.

The sample set may be stored in a sample set model store, while injection raw data blobs may be sent to a separate acquired data raw blob store.

The acquisition device 326 may also generate metadata describing the configuration of the acquisition device 326, details of the experiment being performed, a decoder configured to decode data generated for the experiment, etc. This metadata may be stored in a metadata catalog 130. As with the raw data store 124, the metadata catalog 130 may store metadata associated with multiple different acquisition devices in multiple different configurations.

The raw data may be decodable by a set of decoders 304, where each decoder is associated with a particular data type. For example, the decoder may be associated with a particular type of raw data generated by a chromatography instrument in a specific acquisition mode. That instrument may output a stream of raw data, including (e.g.) binary data, arrays of information, etc. The decoder may be programmed to parse a stream of raw data generated by such an instrument so that the data stream can be meaningfully interpreted.

In some embodiments, a single decoder may be associated with multiple data types; in further embodiments, multiple versions of the same decoder may each be associated with different data types. The decoders 304 may be embedded within a data service 302, such as an autonomous service (e.g., via reflection).

Each of the autonomous services may expose one or more endpoint interfaces. A particular decoder may be associated with each endpoint interface. The decoder may be configured to interpret the raw data that is associated with the endpoint interface.

For example, these endpoints may Representation State Transfer (REST) endpoints capable of receiving RESTful Application Programming Interface (API) calls. An endpoint interface may receive a request for raw data acquired by a chromatography instrument. The data ecosystem 318 may expose multiple endpoint interfaces; for example, each autonomous service may be associated with and may expose at least one endpoint interface. An application 310a, 310c configured to process the raw data may call into the endpoint interface using an API call in order to retrieve the data.

The autonomous service (or another construct) may retrieve the requested raw data from a raw data store, apply the decoder to the raw data to generate decoded data, and may return the decoded data in response to the original request. For example, the autonomous service may apply the decoder to the raw data and provide decoded data to the requesting application, or the autonomous service may identify the decoder and provide it (or a location at which it can be accessed) to the requesting application along with the raw data (or a location of the raw data). In the latter case, the application may decode the data with the decoder.

Returning to the above described example, the autonomous service may retrieve the sample set models from the sample set model store and/or may retrieve the raw data blobs from the raw data blob store. The data may be decoded according to the decoder, and either version of the data (the raw data blobs or the sample set) may be provided to the application. The reason for supplying either or both of the raw data blobs and the sample set models is that the application may be tuned, for performance reasons, to use one or the other representation of the data.

By exposing the endpoint interfaces in this way, an application 310a, 310c can request data acquired by a chromatography instrument without needing to understand how to interpret the data. Furthermore, an application 310a, 310c may deposit the data in a known or common format into a central repository along with metadata indicating, e.g., when the data was received by the application, when the data was processed by the decoder, the identity of user who captured the data, the identity of the instrument that generated the data, and other information describing how and when the data was acquired. Accordingly, when new types of instruments are brought online (potentially outputting data in a different streaming format), it is not necessary to reprogram each application 310a, 310c that might use that data. Because each application 310a, 310c need not be programmed with specifics of how to interpret each different type of data stream, more different types of data can be made available to the applications, which allows for more complex analyses. This configuration also allows multiple different types of data to be stored together in a common source structure, simplifying data retrieval and storage.

In the depicted embodiment, the endpoint interfaces are of two types. A first type serves as a catalog endpoint 306, which is configured to receive requests for metadata. In response to receiving a request for metadata on the catalog endpoint 306, the data service 302 may identify the corresponding metadata in the metadata catalog 130. The data service 302 may then either return the requested metadata to the requesting application, or may return the location of the metadata so that it can be retrieved by the application as needed.

Another type of endpoint interface may serve as a data endpoint 308. There are generally a number of data endpoints 308 in the data ecosystem 312 corresponding to a number of data types that the raw data store 124 is capable of supporting. Each data endpoint 308 is characterized by a data type. When an application requests data, it may call into the raw data store or the metadata catalog to identify the type of the data; for example, the data may be tagged with a codec key that is stored with the data and/or in the metadata. The endpoint interfaces may be callable based on the data type, so once the data type is known the requesting application may identify the appropriate endpoint interface to decode the data and may formulate an appropriate RESTful API call to communicate with the interface. This provides an efficient way for the application to identify and call into the autonomous service that is capable of decoding the data.

Consequently, incoming requests are separated into metadata-specific requests and data-specific requests. Each is handled by a different type of endpoint. This helps to segregate incoming requests and provides requesting applications with a known endpoint to target for appropriate types of requests.

In this example, a single autonomous service handles requests for metadata and each different data type. Although straightforward to implement, it may be necessary to update the entire autonomous service every time one of the data types is changed, or a new data type is added. This can cause unnecessary downtime. Furthermore, the autonomous service needs to be capable of accessing both the metadata catalog 130 and the raw data store 124. These issues can be alleviated by dividing responsibility for different tasks between different autonomous services. An example of such an environment is described next in connection with FIG. 3B.

FIG. 3B illustrates an alternative configuration in which (1) metadata requests are all directed to a particular data service 314, which interfaces with the document store 128 but not the raw data store 124, and (2) data requests are submitted to any of a number of additional autonomous services, each of which has a particular decoder or set of decoders embedded and handles requests specific to the data type of its embedded decoders.

In this configuration, multiple data services 320, 322, etc. service incoming requests for data. Furthermore, at least one data service 314 is specifically configured to respond to requests for metadata. The data service 314 responding to metadata requests does not respond to requests for data, and accordingly does not need to implement any functionality related to the decoders. Similarly, the data services 320, 322, etc. responding to data requests do not need to implement any of the functionality for querying the metadata catalog. When new data types are added, a new autonomous service implementing the decoder for the new data type may be added, or an existing autonomous service may be updated with the new functionality. Meanwhile, most of the autonomous services can remain unchanged. Similarly, if the metadata catalog API is ever changed, only the metadata-handling autonomous service needs to be updated.

The raw data raw data store 124 includes data of multiple different data types. Collectively, the autonomous services may be configured to decode each of the plurality of different data types. For example, the multiple different data types may be included in an interface specification, which may describe how to decode the various different types. The interface specification may be capable of being implemented, at least in part, by each of the autonomous services by implementing corresponding data endpoints 308 and decoders 316, 324. Each data service 320, 322 may be associated with a different set of decoders 316, 324, although there may be some overlap in the decoders supported by different data services. However, no single data service implements all of the decoders, so the functionality for decoding different types of data is distributed across multiple data services. Therefore, different parts of the interface specification may be split between multiple different autonomous services, so that each implements a part, but not all, of the interface specification. Each part of the interface specification may be implemented by at least one of the autonomous services so that, collectively, the group of interface services implements the interface specification.

Because each autonomous service is tasked with only implementing a portion of the interface specification, each autonomous service can be made simpler (since it need not be concerned with providing decoders and endpoint interfaces for portions of the interface specification that it does not implement). New autonomous services can be easily added to deal with new capabilities, and it is not necessary to take down all of the autonomous services when one decoder needs to be updated.

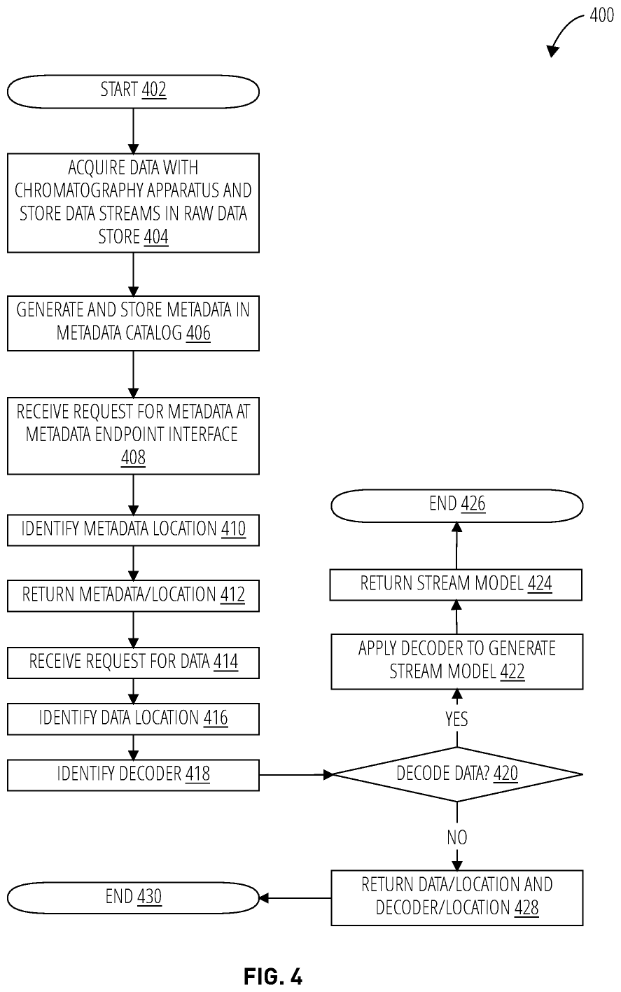

FIG. 4 depicts exemplary data retrieval logic 400 for storing data in, and retrieving data from, a chromatography data processing environment, according to an exemplary embodiment. The data retrieval logic 400 may be embodied as a computer-implemented method or as instructions stored on a non-transitory computer-readable storage medium, and may be configured to cause a processor to perform the logical blocks included in FIG. 4. In some embodiments, the data retrieval logic 400 may be performed by a computing system configured to perform the logical blocks included in FIG. 4.

Processing starts at block 402. At block 404, a chromatography apparatus may acquire data. For instance, the chromatography apparatus may perform an experiment and output data in the form of a stream of measurements. The chromatography apparatus may store the measurements in a raw data store. At block 406, the chromatography apparatus may generate metadata related to the experiment and may store the metadata in a metadata catalog distinct from the raw data store.

At block 408, a request for metadata may be received at a metadata endpoint interface. The metadata endpoint interface may be in direct communication with, and associated with, an autonomous service. The metadata endpoint interface may expose an API and may receive the request via a RESTful API call. The request may be received from an application configured to process raw chromatography data.

At block 410, the autonomous service associated with the metadata endpoint interface that received the request may identify a location of the requested metadata. For example, the autonomous service may query the metadata catalog 130 based on the request to identify where in the metadata catalog 130 the metadata is stored. At block 412, the system may return the metadata or the location of the metadata to the requesting application.

At block 414, a request for data may be received at a data endpoint interface. The data endpoint interface may be in direct communication with, and associated with, an autonomous service. The autonomous service may or may not be the same autonomous service as the one associated with the metadata endpoint interface. The data endpoint interface may expose an API and may receive the request via a RESTful API call. The request may be received from an application configured to process raw chromatography data, and may be the same application as the one that transmitted the request at block 408. The data endpoint interface may be associated with a particular type of data, and the application may generate the RESTful API call based on the type of data to be retrieved. The data endpoint interface may be associated with a decoder in the autonomous service that is capable of decoding the requested data.

At block 416, the autonomous service may identify a location of the requested data. For example, the autonomous service may query the raw data store 124 based on the request to identify where in the raw data store 124 the requested data (which may be in the form of a stream) is located.

The data endpoint interface that received the request for the data may be associated with a decoder in the autonomous service that is capable of decoding the requested data. At block 418, the decoder associated with the data endpoint interface may be identified and its location may be ascertained (e.g., a hyperlink to a location of the decoder may be generated).

At decision block 420, the autonomous service determines if it is configured to decode the data. In some embodiments, the autonomous service is configured to apply the decoder identified at block 418 to parse the data stream identified at block 416. In other embodiments, the autonomous service simply identifies the location of the raw data and the decoder, and provides those locations to the application (which can then decode the data itself).

If the decision at decision block 420 is “YES,” then processing proceeds to block 422 and the autonomous service applies the identified decoder to decode the the identified raw data. This may generate a stream model, which is provided as an output to the requesting application at block 424. Processing then proceeds to done block 426 and terminates.

If the decision at decision block 420 is “NO,” (i.e., the autonomous service is not configured to decode the data), then at block 428 the autonomous service returns the location of the data identified at block 416 and the location of the decoder identified at block 418. Processing may then proceed to done block 430 and terminate.

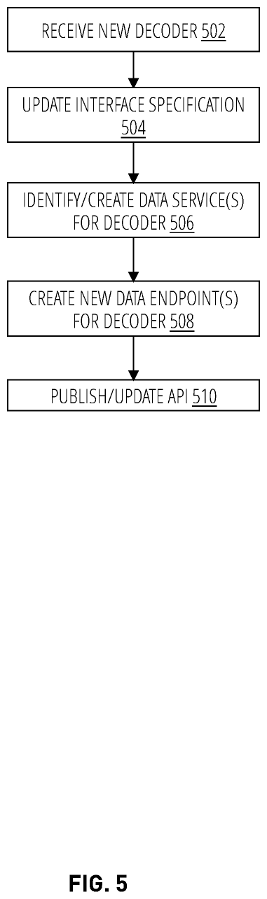

FIG. 5 is a flowchart depicting exemplary logic for adding a new decoders 304 to a data processing environment, such as the ones depicted in FIG. 3A and FIG. 3B.

At block 502, a new decoder for the data processing environment may be received. The decoder may be received in connection with a new type of data becoming supportable by the data processing environment. Receiving the decoder may include, for example, receiving a location from which the decoder may be retrieved, or receiving computer-executable code for the decoder.

The decoder may include logic to decode (e.g., parse) a stream of data from an MS instrument. The decoder may be associated with an identifier, and the identifier may contain metadata sufficient to identify a data source for the decoder, data format, data type, decoder type, or decoder parameters such as a file type extension, description of the data, header information, etc.

At block 504, the interface specification for the data processing environment (e.g., a document describing the data types supported by the environment and/or the structure of the data services that can be used to decode the data) may be updated based on the information received at block 502. As previously noted, in some embodiments each data service implements the entire interface specification; in others, each data service implements a part of the interface specification so that, between all the available data services, the interface specification is implemented in full.

Depending on which embodiment is being used, at block 506 the system may identify an existing data service or services to implement the decoder, or may create a new data service for the decoder. When each data service implements all the decoders, then all the data services may be identified at block 506. If only one or a subset of the data services will implement the decoder, then a new data service or services may be created (and/or one or more existing data services may be identified to support the new decoder). In some embodiments, the decoder may be loaded into the data service by reflection to embed the decoder within the data service.

At block 508, one or more new data endpoints corresponding to the decoder may be created on the data services identified or created at block 506. This may involve creating a new interface to the data service capable of receiving API calls. In some embodiments, new RESTful endpoints may be created.

At block 510, the system may optionally update the specification for the API exposed by the data service(s) and publish it to end users. The API specification may be updated to describe how to call the new decoder via the endpoint(s) created at block 508, such as by providing a method name or path for calling into the endpoint. In some embodiments, it is not necessary to advertise the API specification, because the API call may be patterned based on the data type of the decoder associated with the endpoint. Accordingly, applications may be capable of deriving or inferring the proper API to be called based on the type of the data being requested.

FIG. 6 illustrates one example of a system architecture and data processing device that may be used to implement one or more illustrative aspects described herein in a standalone and/or networked environment. Various network nodes, such as the data server 610, web server 606, computer 604, and laptop 602 may be interconnected via a wide area network 608 (WAN), such as the internet. Other networks may also or alternatively be used, including private intranets, corporate networks, LANs, metropolitan area networks (MANs) wireless networks, personal networks (PANs), and the like. Network 608 is for illustration purposes and may be replaced with fewer or additional computer networks. A local area network (LAN) may have one or more of any known LAN topology and may use one or more of a variety of different protocols, such as ethernet. Devices data server 610, web server 606, computer 604, laptop 602 and other devices (not shown) may be connected to one or more of the networks via twisted pair wires, coaxial cable, fiber optics, radio waves or other communication media.

Computer software, hardware, and networks may be utilized in a variety of different system environments, including standalone, networked, remote-access (aka, remote desktop), virtualized, and/or cloud-based environments, among others.

The term “network” as used herein and depicted in the drawings refers not only to systems in which remote storage devices are coupled together via one or more communication paths, but also to stand-alone devices that may be coupled, from time to time, to such systems that have storage capability. Consequently, the term “network” includes not only a “physical network” but also a “content network,” which is comprised of the data—attributable to a single entity—which resides across all physical networks.

The components may include data server 610, web server 606, and client computer 604, laptop 602. Data server 610 provides overall access, control and administration of databases and control software for performing one or more illustrative aspects described herein. Data serverdata server 610 may be connected to web server 606 through which users interact with and obtain data as requested. Alternatively, data server 610 may act as a web server itself and be directly connected to the internet. Data server 610 may be connected to web server 606 through the network 608 (e.g., the internet), via direct or indirect connection, or via some other network. Users may interact with the data server 610 using remote computer 604, laptop 602, e.g., using a web browser to connect to the data server 610 via one or more externally exposed web sites hosted by web server 606. Client computer 604, laptop 602 may be used in concert with data server 610 to access data stored therein, or may be used for other purposes. For example, from client computer 604, a user may access web server 606 using an internet browser, as is known in the art, or by executing a software application that communicates with web server 606 and/or data server 610 over a computer network (such as the internet).

Servers and applications may be combined on the same physical machines, and retain separate virtual or logical addresses, or may reside on separate physical machines. FIG. 6 illustrates just one example of a network architecture that may be used, and those of skill in the art will appreciate that the specific network architecture and data processing devices used may vary, and are secondary to the functionality that they provide, as further described herein. For example, services provided by web server 606 and data server 610 may be combined on a single server.

Each component data server 610, web server 606, computer 604, laptop 602 may be any type of known computer, server, or data processing device. Data server 610, e.g., may include a processor 612 controlling overall operation of the data server 610. Data server 610 may further include RAM 616, ROM 618, network interface 614, input/output interfaces 620 (e.g., keyboard, mouse, display, printer, etc.), and memory 622. Input/output interfaces 620 may include a variety of interface units and drives for reading, writing, displaying, and/or printing data or files. Memory 622 may further store operating system software 624 for controlling overall operation of the data server 610, control logic 626 for instructing data server 610 to perform aspects described herein, and other application software 628 providing secondary, support, and/or other functionality which may or may not be used in conjunction with aspects described herein. The control logic may also be referred to herein as the data server software control logic 626. Functionality of the data server software may refer to operations or decisions made automatically based on rules coded into the control logic, made manually by a user providing input into the system, and/or a combination of automatic processing based on user input (e.g., queries, data updates, etc.).

Memory 1122 may also store data used in performance of one or more aspects described herein, including a first database 632 and a second database 630. In some embodiments, the first database may include the second database (e.g., as a separate table, report, etc.). That is, the information can be stored in a single database, or separated into different logical, virtual, or physical databases, depending on system design. Web server 606, computer 604, laptop 602 may have similar or different architecture as described with respect to data server 610. Those of skill in the art will appreciate that the functionality of data server 610 (or web server 606, computer 604, laptop 602) as described herein may be spread across multiple data processing devices, for example, to distribute processing load across multiple computers, to segregate transactions based on geographic location, user access level, quality of service (QoS), etc.

One or more aspects may be embodied in computer-usable or readable data and/or computer-executable instructions, such as in one or more program modules, executed by one or more computers or other devices as described herein. Generally, program modules include routines, programs, objects, components, data structures, etc. that perform particular tasks or implement particular abstract data types when executed by a processor in a computer or other device. The modules may be written in a source code programming language that is subsequently compiled for execution, or may be written in a scripting language such as (but not limited to) HTML or XML. The computer executable instructions may be stored on a computer readable medium such as a nonvolatile storage device. Any suitable computer readable storage media may be utilized, including hard disks, CD-ROMs, optical storage devices, magnetic storage devices, and/or any combination thereof. In addition, various transmission (non-storage) media representing data or events as described herein may be transferred between a source and a destination in the form of electromagnetic waves traveling through signal-conducting media such as metal wires, optical fibers, and/or wireless transmission media (e.g., air and/or space). various aspects described herein may be embodied as a method, a data processing system, or a computer program product. Therefore, various functionalities may be embodied in whole or in part in software, firmware and/or hardware or hardware equivalents such as integrated circuits, field programmable gate arrays (FPGA), and the like. Particular data structures may be used to more effectively implement one or more aspects described herein, and such data structures are contemplated within the scope of computer executable instructions and computer-usable data described herein.

The components and features of the devices described above may be implemented using any combination of discrete circuitry, application specific integrated circuits (ASICs), logic gates and/or single chip architectures. Further, the features of the devices may be implemented using microcontrollers, programmable logic arrays and/or microprocessors or any combination of the foregoing where suitably appropriate. It is noted that hardware, firmware and/or software elements may be collectively or individually referred to herein as “logic” or “circuit.”

It will be appreciated that the exemplary devices shown in the block diagrams described above may represent one functionally descriptive example of many potential implementations. Accordingly, division, omission or inclusion of block functions depicted in the accompanying figures does not infer that the hardware components, circuits, software and/or elements for implementing these functions would be necessarily be divided, omitted, or included in embodiments.

At least one computer-readable storage medium may include instructions that, when executed, cause a system to perform any of the computer-implemented methods described herein.

Some embodiments may be described using the expression “one embodiment” or “an embodiment” along with their derivatives. These terms mean that a particular feature, structure, or characteristic described in connection with the embodiment is included in at least one embodiment. The appearances of the phrase “in one embodiment” in various places in the specification are not necessarily all referring to the same embodiment. Moreover, unless otherwise noted the features described above are recognized to be usable together in any combination. Thus, any features discussed separately may be employed in combination with each other unless it is noted that the features are incompatible with each other.

With general reference to notations and nomenclature used herein, the detailed descriptions herein may be presented in terms of program procedures executed on a computer or network of computers. These procedural descriptions and representations are used by those skilled in the art to most effectively convey the substance of their work to others skilled in the art.

A procedure is here, and generally, conceived to be a self-consistent sequence of operations leading to a desired result. These operations are those requiring physical manipulations of physical quantities. Usually, though not necessarily, these quantities take the form of electrical, magnetic or optical signals capable of being stored, transferred, combined, compared, and otherwise manipulated. It proves convenient at times, principally for reasons of common usage, to refer to these signals as bits, values, elements, symbols, characters, terms, numbers, or the like. It should be noted, however, that all of these and similar terms are to be associated with the appropriate physical quantities and are merely convenient labels applied to those quantities.

Further, the manipulations performed are often referred to in terms, such as adding or comparing, which are commonly associated with mental operations performed by a human operator. No such capability of a human operator is necessary, or desirable in most cases, in any of the operations described herein, which form part of one or more embodiments. Rather, the operations are machine operations. Useful machines for performing operations of various embodiments include general purpose digital computers or similar devices.

Some embodiments may be described using the expression “coupled” and “connected” along with their derivatives. These terms are not necessarily intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Various embodiments also relate to apparatus or systems for performing these operations. This apparatus may be specially constructed for the required purpose or it may comprise a general purpose computer as selectively activated or reconfigured by a computer program stored in the computer. The procedures presented herein are not inherently related to a particular computer or other apparatus. Various general purpose machines may be used with programs written in accordance with the teachings herein, or it may prove convenient to construct more specialized apparatus to perform the required method steps. The required structure for a variety of these machines will appear from the description given.

It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment. In the appended claims, the terms “including” and “in which” are used as the plain-English equivalents of the respective terms “comprising” and “wherein,” respectively. Moreover, the terms “first,” “second,” “third,” and so forth, are used merely as labels, and are not intended to impose numerical requirements on their objects.

What has been described above includes examples of the disclosed architecture. It is, of course, not possible to describe every conceivable combination of components and/or methodologies, but one of ordinary skill in the art may recognize that many further combinations and permutations are possible. Accordingly, the novel architecture is intended to embrace all such alterations, modifications and variations that fall within the spirit and scope of the appended claims.

What is claimed is:

1. A method comprising:
receiving, at an endpoint interface of an autonomous service in a chromatography data processing environment, a request for raw data acquired by a chromatography instrument, where the environment exposes multiple endpoint interfaces;
identifying a decoder configured to interpret the raw data that is associated with the endpoint interface;
retrieving the requested raw data from a raw data store; and
applying the decoder to the raw data to generate decoded data.

2. The method of claim 1, wherein the autonomous service is a first autonomous service and is configured to interface with the raw data store, and further comprising a second autonomous service configured to interface with a metadata catalog storing metadata that describes how the raw data was acquired.

3. The method of claim 1, wherein the endpoint interface is a first endpoint interface and is configured to receive requests for raw data, and further comprising a second endpoint interface configured to receive requests for metadata that describes how the raw data was acquired.

4. The method of claim 1, wherein the decoder is associated with multiple different endpoint interfaces.

5. The method of claim 1, wherein the raw data store includes data that originates in at least one of:
multiple different chromatography apparatuses, or
chromatography apparatuses operating in multiple different acquisition modes.

6. The method of claim 1, wherein:
the raw data store includes data of a plurality of different data types,
the autonomous service is one of a plurality of autonomous services; and
the plurality of autonomous services are collectively configured to decode the plurality of different data types.

7. The method of claim 6, wherein the plurality of autonomous services collectively include a plurality of endpoint interfaces, each endpoint interface characterized by a data type.

8. A non-transitory computer-readable storage medium, the computer-readable storage medium including instructions that when executed by a computer, cause the computer to:
receive, at an endpoint interface of an autonomous service in a chromatography data processing environment, a request for raw data acquired by a chromatography instrument, where the environment exposes multiple endpoint interfaces;
identify a decoder configured to interpret the raw data that is associated with the endpoint interface;
retrieve the requested raw data from a raw data store; and
apply the decoder to the raw data to generate decoded data.

9. The computer-readable storage medium of claim 8, wherein the autonomous service is a first autonomous service and is configured to interface with the raw data store, and wherein the instructions further configure the computer to a second autonomous service configured to interface with a metadata catalog store metadata that describes how the raw data was acquired.

10. The computer-readable storage medium of claim 8, wherein the endpoint interface is a first endpoint interface and is configured to receive requests for raw data, and wherein the instructions further configure the computer to a second endpoint interface configured to receive requests for metadata that describes how the raw data was acquired.

11. The computer-readable storage medium of claim 8, wherein the decoder is associated with multiple different endpoint interfaces.

12. The computer-readable storage medium of claim 8, wherein the raw data store includes data that originates in at least one of:
multiple different chromatography apparatuses, or
chromatography apparatuses operate in multiple different acquisition modes.

13. The computer-readable storage medium of claim 8, wherein:
the raw data store includes data of a plurality of different data types,
the autonomous service is one of a plurality of autonomous services; and
the plurality of autonomous services are collectively configured to decode the plurality of different data types.

14. The computer-readable storage medium of claim 13, wherein the plurality of autonomous services collectively include a plurality of endpoint interfaces, each endpoint interface characterized by a data type.

15. A computing apparatus comprising:
a processor; and
a memory storing instructions that, when executed by the processor, configure the apparatus to:
receive, at an endpoint interface of an autonomous service in a chromatography data processing environment, a request for raw data acquired by a chromatography instrument, where the environment exposes multiple endpoint interfaces;
identify a decoder configured to interpret the raw data that is associated with the endpoint interface;
retrieve the requested raw data from a raw data store;
apply the decoder to the raw data to generate decoded data.

16. The computing apparatus of claim 15, wherein the autonomous service is a first autonomous service and is configured to interface with the raw data store, and wherein the instructions further configure the apparatus to a second autonomous service configured to interface with a metadata catalog store metadata that describes how the raw data was acquired.

17. The computing apparatus of claim 15, wherein the endpoint interface is a first endpoint interface and is configured to receive requests for raw data, and wherein the instructions further configure the apparatus to a second endpoint interface configured to receive requests for metadata that describes how the raw data was acquired.

18. The computing apparatus of claim 15, wherein the decoder is associated with multiple different endpoint interfaces.

19. The computing apparatus of claim 15, wherein the raw data store includes data that originates in at least one of:
multiple different chromatography apparatuses, or
chromatography apparatuses operate in multiple different acquisition modes.

20. The computing apparatus of claim 15, wherein:
the raw data store includes data of a plurality of different data types,
the autonomous service is one of a plurality of autonomous services; and
the plurality of autonomous services are collectively configured to decode the plurality of different data types.

21. The computing apparatus of claim 20, wherein the plurality of autonomous services collectively include a plurality of endpoint interfaces, each endpoint interface characterized by a data type.
